(19) United States
(12) Patent Application Publication
(10) Pub. No.: US 2023/0371132 A1
(43) Pub. Date: Nov. 16, 2023

(54) HEATING BODY, VAPORIZATION ASSEMBLY, AND ELECTRONIC VAPORIZATION DEVICE

(71) Applicant: SHENZHEN SMOORE TECHNOLOGY LIMITED, Shenzhen (CN)

(72) Inventors: Ming LV, Shenzhen (CN); Wei WANG, Shenzhen (CN); Mingda ZHU, Shenzhen (CN); Rongji HUANG, Shenzhen (CN); Jianming FENG, Shenzhen (CN)

(21) Appl. No.: 18/091,175

(22) Filed: Dec. 29, 2022

Related U.S. Application Data
(63) Continuation of application No. PCT/CN2022/092859, filed on May 13, 2022.

(51) Int. Cl. H05B 3/16

(52) U.S. Cl. CPC H05B 3/16; A61M 11/044

(57) ABSTRACT

A heating body for an electronic vaporization is disclosed. The heating body comprises a liquid guiding substrate, a heating material layer, a first protective film, and a second protective film. The liquid guiding substrates comprises a heating region and an electrode region. The heating material layer is arranged on a first surface of the liquid guiding substrate. The first protective film is made of a non-conductive material resistant to a corrosion of an aerosol-generation substrate. The second protective film is made of a conductive material resistant to a corrosion of the aerosol-generation substrate.

### CROSS-REFERENCE TO RELATED APPLICATION

This application is a continuation of International Application No. PCT/CN2022/092859, filed on May 13, 2022, the entire disclosure of which is hereby incorporated by reference.

### TECHNICAL FIELD

This application relates to the field of vaporization technologies, and in particular, to a heating body, a vaporization assembly, and an electronic vaporization device.

### BACKGROUND

A typical electronic vaporization device is formed by components such as a heating body, a battery, and a control circuit. The heating body is a core component of the electronic vaporization device, and characteristics thereof decide a vaporization effect and use experience of the electronic vaporization device.

An existing heating body has a risk of being corroded in a strong corrosive aerosol-generation substrate and has a relatively short service life.

### SUMMARY

In view of this, this application provides a heating body, a vaporization assembly, and an electronic vaporization device, to resolve the technical problem that a service life of a heating body is relatively short in the related art.

To resolve the foregoing technical problem, a first technical solution provided in this application is to provide a heating body, applicable to an electronic vaporization device and configured to vaporize an aerosol-generation substrate, the heating body including a liquid guiding substrate, a heating material layer, a first protective film, and a second protective film, where the liquid guiding substrate includes a heating region and an electrode region; the heating material layer is arranged on a first surface of the liquid guiding substrate; the heating material layer is a resistance heating material and includes a heating portion arranged in the heating region and a connection portion arranged in the electrode region; the first protective film is at least partially arranged on a surface of the heating portion that is away from the liquid guiding substrate; a material of the first protective film is a non-conductive material resistant to corrosion of the aerosol-generation substrate; the second protective film is at least partially arranged on a surface of the connection portion that is away from the liquid guiding substrate; and a material of the second protective film is a conductive material resistant to corrosion of the aerosol-generation substrate.

In an implementation, the material of the first protective film is ceramic or glass.

In an implementation, the material of the first protective film is the ceramic; and a material of the ceramic is one or more of aluminum nitride, silicon nitride, aluminum oxide, silicon oxide, silicon carbide, or zirconium oxide.

In an implementation, a thickness of the first protective film ranges from 10 nm to 1000 nm.

In an implementation, the material of the second protective film is conductive ceramic or metal.

In an implementation, the material of the second protective film is the conductive ceramic, and a material of the conductive ceramic is one or more of titanium nitride or titanium diboride.

In an implementation, a thickness of the second protective film ranges from 10 nm to 2000 nm.

In an implementation, the liquid guiding substrate is a dense liquid guiding substrate; and the liquid guiding substrate further includes a second surface arranged opposite to the first surface, the liquid guiding substrate includes a plurality of first micropores, and the plurality of first micropores are ordered through holes running through the first surface and the second surface.

In an implementation, a material of the liquid guiding substrate is quartz, glass, or dense ceramic, and the plurality of first micropores are straight through holes.

In an implementation, a material of the liquid guiding substrate is porous ceramic, and the liquid guiding substrate includes a plurality of disordered through holes; or
the liquid guiding substrate includes a porous ceramic layer and a dense ceramic layer that are stacked, the dense ceramic layer includes a plurality of ordered straight through holes perpendicular to a thickness direction of the liquid guiding substrate; and the heating material layer is arranged on a surface of the dense ceramic layer that is away from the porous ceramic layer.

In an implementation, the heating material layer is a heating film, and a thickness of the heating film ranges from 200 nm to 5 μm.

In an implementation, a resistivity of the heating material layer is less than 0.06*10⁻⁶ Ω·m.

In an implementation, a material of the heating material layer is aluminum, copper, silver, gold, nickel, chromium, platinum, titanium, zirconium, palladium, iron, or alloy thereof.

In an implementation, a thickness of the heating material layer ranges from 5 μm to 100 μm, and the heating material layer is a printed metal slurry layer.

In an implementation, the liquid guiding substrate is in a shape of a flat plate, an arc, or a barrel.

In an implementation, the first protective film covers the entire heating portion, and the second protective film covers the entire connection portion.

In an implementation, the liquid guiding substrate is in a shape of a cylinder, the liquid guiding substrate includes an inner surface and an outer surface, and the heating material layer is arranged on the inner surface or the outer surface.

In an implementation, the heating material layer, the first protective film, and the second protective film are formed on the first surface of the liquid guiding substrate in a physical vapor deposition or chemical vapor deposition manner.

In an implementation, the connection portion of the heating material layer and the second protective film form an electrode.

In an implementation, the plurality of first micropores are straight through holes, and the heating material layer and the first protective film extend into a wall surface of each of the plurality of first micropores.

To resolve the foregoing technical problem, a second technical solution provided in this application is to provide a vaporization assembly, including a liquid storage cavity and a heating body, where the liquid storage cavity is configured to store a liquid aerosol-generation substrate; the heating body is the heating body according to any one of the foregoing; and the heating body is in fluid communication with the liquid storage cavity.

To resolve the foregoing technical problem, a third technical solution provided in this application is to provide an electronic vaporization device, including a vaporization assembly and a power supply assembly, where the vaporization assembly is the vaporization assembly according to the foregoing, and the power supply assembly is electrically connected to the heating body.

Beneficial effects of this application are as follows: different from the related art, this application discloses a heating body, a vaporization assembly, and an electronic vaporization device. The heating body includes a liquid guiding substrate, a heating material layer, a first protective film, and a second protective film, where the liquid guiding substrate includes a heating region and an electrode region; the heating material layer is arranged on a first surface of the liquid guiding substrate and includes a heating portion arranged in the heating region and a connection portion arranged in the electrode region; the first protective film is arranged on a surface of the heating portion that is away from the liquid guiding substrate; a material of the first protective film is a non-conductive material resistant to corrosion of an aerosol-generation substrate; the second protective film is arranged on a surface of the connection portion that is away from the liquid guiding substrate; and a material of the second protective film is a conductive material resistant to corrosion of the aerosol-generation substrate. By protecting the heating region and the electrode region of the heating material layer through different protective films, the heating material layer is prevented from being corroded by the aerosol-generation substrate, thereby helping improve a service life of the heating material layer.

### BRIEF DESCRIPTION OF THE DRAWINGS

To describe the technical solutions in the embodiments of this application more clearly, the following briefly introduces the accompanying drawings required for describing the embodiments. Apparently, the accompanying drawings in the following description show merely some embodiments of this application, and a person of ordinary skill in the art may still derive other accompanying drawings from these accompanying drawings without creative efforts.

FIG. 1 is a schematic structural diagram of an electronic vaporization device according to an embodiment of this application;

FIG. 2 is a schematic structural diagram of a vaporization assembly of an electronic vaporization device according to an embodiment of this application;

FIG. 3a is a schematic structural diagram of a first implementation of a heating body according to this application;

FIG. 3b is a schematic top structural view of the heating body provided in FIG. 3a;

FIG. 4 is a schematic structural diagram of a liquid guiding substrate of the heating body provided in FIG. 3a;

FIG. 5 is a schematic structural diagram of a second implementation of a heating body according to this application;

FIG. 6 is a schematic structural diagram of a third implementation of a heating body according to this application;

FIG. 7 is a schematic structural diagram of a fourth implementation of a heating body according to this application;

FIG. 8 is a schematic structural diagram of a fifth implementation of a heating body according to this application;

FIG. 9 is a schematic structural diagram of a sixth implementation of a heating body according to this application; and

FIG. 10 is a schematic diagram of wet combustion on a heating body according to this application.

### DETAILED DESCRIPTION

The technical solutions in the embodiments of this application are clearly and completely described below with reference to the accompanying drawings in the embodiments of this application. Apparently, the described embodiments are merely some rather than all of the embodiments of this application. All other embodiments obtained by a person of ordinary skill in the art based on the embodiments of this application without creative efforts shall fall within the protection scope of this application.

In the following description, for the purpose of illustration rather than limitation, specific details such as the specific system structure, interface, and technology are proposed to thoroughly understand this application.

The terms “first”, “second”, and “third” in this application are merely intended for a purpose of description, and shall not be understood as indicating or implying relative significance or implicitly indicating the number of indicated technical features. Therefore, features defining “first”, “second”, and “third” can explicitly or implicitly include at least one of the features. In the description of this application, “a plurality of” means at least two, such as two and three unless it is specifically defined otherwise. All directional indications (for example, upper, lower, left, right, front, and rear) in the embodiments of this application are only used for explaining relative position relationships, movement situations, or the like between various components in a specific posture (as shown in the accompanying drawings). If the specific posture changes, the directional indications change accordingly. In the embodiments of this application, the terms “include”, “have”, and any variant thereof are intended to cover a non-exclusive inclusion. For example, a process, method, system, product, or device that includes a series of steps or units is not limited to the listed steps or units, but further optionally includes a step or unit that is not listed, or further optionally includes another step or component that is intrinsic to the process, method, product, or device.

“Embodiment” mentioned in this specification means that particular features, structures, or characteristics described with reference to the embodiment may be included in at least one embodiment of this application. The term appearing at different positions of this specification may not refer to the same embodiment or an independent or alternative embodiment that is mutually exclusive with another embodiment. A person skilled in the art explicitly or implicitly understands that the embodiments described in this specification may be combined with other embodiments.

This application is described in detail below with reference to the accompanying drawings and the embodiments.

Referring to FIG. 1, FIG. 1 is a schematic structural diagram of an electronic vaporization device according to an embodiment of this application.

In this embodiment, an electronic vaporization device 100 is provided. The electronic vaporization device 100 may be configured to vaporize an aerosol-generation substrate. The electronic vaporization device 100 includes a vaporization assembly 1 and a power supply assembly 2 that are electrically connected to each other.

The vaporization assembly 1 is configured to store an aerosol-generation substrate and vaporize the aerosol-generation substrate to form aerosols that can be inhaled by a user. The vaporization assembly 1 specifically may be applicable to different fields such as medical care, cosmetology, and recreation inhalation. In a specific embodiment, the vaporization assembly 1 may be applicable to an electronic aerosol vaporization device to vaporize an aerosol-generation substrate and generate aerosols for inhalation by an inhaler, and the following embodiments are described by using the recreation inhalation as an example.

For a specific structure and functions of the vaporization assembly 1, reference may be made to the specific structure and functions of the vaporization assembly 1 involved in the following embodiments, same or similar technical effects may also be implemented, and details are not described herein again.

The power supply assembly 2 includes a battery (not shown in the figure) and a controller (not shown in the figure). The battery is configured to supply electric energy for operation of the vaporization assembly 1, to cause the vaporization assembly 1 to vaporize the aerosol-generation substrate to form aerosols. The controller is configured to control operation of the vaporization assembly 1. The power supply assembly 2 further includes other components such as a battery holder and an airflow sensor.

The vaporization assembly 1 and the power supply assembly 2 may be integrally arranged or may be detachably connected to each other, which may be designed according to a specific requirement.

Power of the electronic vaporization device generally does not exceed 10 W, and the power generally ranges from 6 W to 8.5 W. A voltage of a battery adopted by the electronic vaporization device ranges from 2.5 V to 4.4 V. For a closed electronic vaporization device (an electronic vaporization device into which the user does not need to autonomously inject an aerosol-generation substrate), a voltage of an adopted battery ranges from 3 V to 4.4 V. However, the electronic vaporization device of the present disclosure is not limited to the parameters.

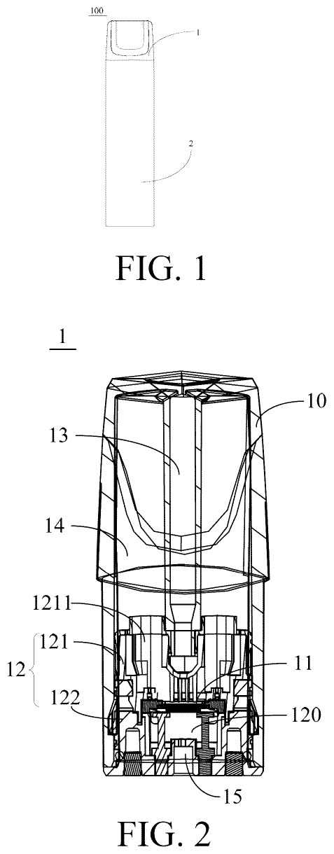

Referring to FIG. 2, FIG. 2 is a schematic structural diagram of a vaporization assembly of an electronic vaporization device according to an embodiment of this application.

The vaporization assembly 1 includes a housing 10, a heating body 11, and a vaporization base 12. The vaporization base 12 includes a mounting cavity (not marked in the figure), and the heating body 11 is arranged in the mounting cavity; and the heating body 11 is arranged together with the vaporization base 12 in the housing 10. The housing 10 is provided with a vapor outlet channel 13, an inner surface of the housing 10, an outer surface of the vapor outlet channel 13, and a top surface of the vaporization base 12 cooperate to form a liquid storage cavity 14, and the liquid storage cavity 14 is configured to store a liquid aerosol-generation substrate. The heating body 11 is electrically connected to the power supply assembly 2, to vaporize the aerosol-generation substrate to generate aerosols.

The vaporization base 12 includes an upper base 121 and a lower base 122, and the upper base 121 and the lower base 122 cooperate to form the mounting cavity; and a vaporization surface of the heating body 11 and a cavity wall of the mounting cavity cooperate to form a vaporization cavity 120. A liquid supplying channel 1211 is provided on the upper base 121, and the liquid supplying channel 1211 is in communication with the mounting cavity. The aerosol-generation substrate in the liquid storage cavity 14 flows into the heating body 11 through the liquid supplying channel 1211, namely, the heating body 11 is in fluid communication with the liquid storage cavity 14. An air inlet channel 15 is provided on the lower base 122, external air enters the vaporization cavity 120 through the air inlet channel 15, carries aerosols vaporized by the heating body 11 to flow to the vapor outlet channel 13, and the user inhales the aerosols through an end opening of the vapor outlet channel 13.

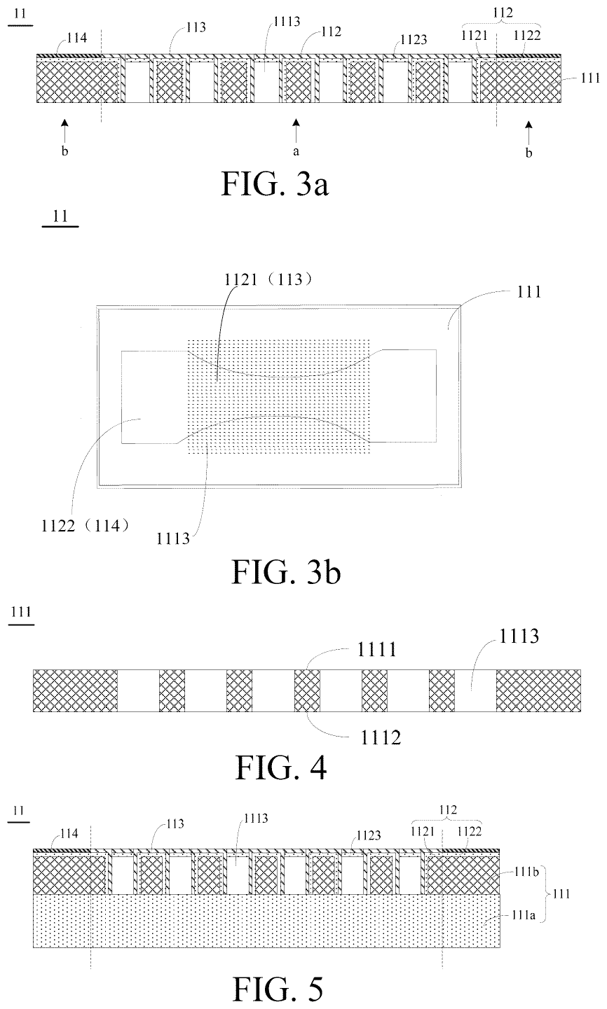

Referring to FIG. 3a to FIG. 4, FIG. 3a is a schematic structural diagram of a first implementation of a heating body according to this application, FIG. 3b is a schematic top structural view of the heating body provided in FIG. 3a, and FIG. 4 is a schematic structural diagram of a liquid guiding substrate of the heating body provided in FIG. 3a.

The heating body 11 includes a liquid guiding substrate 111, a heating material layer 112, a first protective film 113, and a second protective film 114. The liquid guiding substrate 111 plays a role of structure supporting. The heating material layer 112 is a resistance heating material. The liquid guiding substrate 111 includes a first surface 1111 and a second surface 1112 arranged opposite to each other. The first surface 1111 of the liquid guiding substrate 111 includes a heating region a and an electrode region b. The heating material layer 112 is arranged on the first surface 1111 of the liquid guiding substrate 111, and the heating material layer 112 includes a heating portion 1121 arranged in the heating region a and a connection portion 1122 arranged in the electrode region b, where the connection portion 1122 serves as an electrode, and the connection portion 1122 is configured to be electrically connected to the power supply assembly 2. The first protective film 113 is at least partially arranged on a surface of the heating portion 1121 that is away from the liquid guiding substrate 111, and a material of the first protective film 113 is a non-conductive material resistant to corrosion of the aerosol-generation substrate. The second protective film 114 is at least partially arranged on a surface of the connection portion 1122 that is away from the liquid guiding substrate 111, and a material of the second protective film 114 is a conductive material resistant to corrosion of the aerosol-generation substrate.

Through the foregoing arrangement, different regions of the heating material layer 112 are respectively protected by using different protective films, so that corrosion of the aerosol-generation substrate to the heating portion 1121 and the connection portion 1122 is effectively prevented, which helps improve a service life of the heating material layer 112.

Optionally, the first protective film 113 covers the entire heating portion 1121, to prevent corrosion of the aerosol-generation substrate to the entire heating portion 1121, so that the entire heating portion 1121 is protected, which helps improve a service life of the heating body 11.

Optionally, the second protective film 114 covers the entire connection portion 1122, to prevent corrosion of the aerosol-generation substrate to the entire connection portion 1122, so that the entire connection portion 1122 is protected, which helps improve the service life of the heating body 11.

Optionally, an opening (not shown in the figure) is provided on the second protective film 114 to expose a part of the connection portion 1122, and the exposed connection portion 1122 is configured to be in contact with a conductor (not marked in the figure). Through the arrangement, contact resistance between the conductor and the connection portion 1122 is reduced. It may be understood that, the connection portion 1122 is electrically connected to the power supply assembly 2 through the conductor (not marked in the figure); and the conductor may be an ejector pin or a pogo pin.

Optionally, the material of the first protective film 113 is ceramic or glass. Because a material of the heating material layer 112 is metal, a thermal expansion coefficient of ceramic or glass matches the metal heating material layer 112, and adhesion of ceramic or glass matches the metal heating material layer 112. Therefore, ceramic or glass is used as the first protective film 113, and the first protective film 113 can hardly fall off the heating portion 1121, so that the heating portion can be well protected.

When the material of the first protective film 113 is ceramic, the material of the ceramic may be one or more of aluminum nitride, silicon nitride, aluminum oxide, silicon oxide, silicon carbide, or zirconium oxide, which is specifically selected as required. Referring to Table 1 and Table 2, compared with a case that stainless steel resistant to corrosion of the aerosol-generation substrate is used as a protective film to protect the heating portion 1121, ceramic is used as the first protective film 113 to protect the heating portion 1121, and the first protective film 113 includes higher heat conduction performance and a smaller contact angle of the aerosol-generation substrate. The higher heat conduction performance of the first protective film 113 can conduct heat generated by the heating portion 1121 to the aerosol-generation substrate more efficiently, which helps improve the vaporization efficiency of the heating portion 1121; and The smaller contact angle of the first protective film 113 causes the wettability of the aerosol-generation substrate on a surface of the first protective film to be stronger, and the transmission efficiency of the aerosol-generation substrate is higher, which further helps improve the vaporization efficiency of the heating portion 1121. Experiments was performed on the heating body 11 using the first protective film 113 (the first protective film 113 adopts aluminum nitride) as a protective film of the heating portion 1121 and a heating body in the related art (which adopts stainless steel as a protective film), and experiment conditions are as follows: constant power of 6.5 W and inhalation is performed for 3s and then stopped for 27s. A vaporization amount of the heating body 11 provided in this application is 7.2 mg/puff, and a vaporization amount of the heating body in the related art is 6.2 mg/puff, which proves that the vaporization amount may be apparently improved by using ceramic as the first protective film 113. Thermal conductivities of some materials are shown in Table 1; and contact angles of some materials are shown in Table 2.

TABLE 1
Thermal conductivities of materials

| | Protective film | | | | |
|---|---|---|---|---|---|
| | Stainless | Aluminum | Silicon | Aluminum | Silicon |
| Material | steel | nitride | nitride | oxide | carbide |
| Thermal | 15 | 180 | 27 | 45 | 200 |
| conductivity | | | | | |
| (W/m · K) | | | | | |

TABLE 2
Contact angles of materials

| | Stainless | Aluminum | Silicon | Aluminum | Silicon |
|---|---|---|---|---|---|
| Material | steel | nitride | nitride | oxide | carbide |
| Contact angle | 45° | 20° | 20° | 23° | 22° |

Optionally, a thickness of the first protective film 113 ranges from 10 nm to 1000 nm. When the thickness is less than 10 nm, the first protective film 113 can hardly achieve a protection effect, since the density of a thin film is not good, and the aerosol-generation substrate may run through the first protective film 113 to corrode the heating portion 1121; and when the thickness of the first protective film 113 is greater than 1000 nm, stress is excessively great, and as a result, the first protective film 113 is easily cracked due to thermal shock and loses the protection effect.

Optionally, a thickness of the second protective film 114 ranges from 10 nm to 2000 nm. When the thickness is less than 10 nm, the second protective film 114 can hardly achieve a protection effect, since the density of a thin film is not good, and the aerosol-generation substrate may run through the second protective film 114 to corrode the connection portion 1122; and when the thickness of the second protective film 114 is greater than 2000 nm, stress is excessively great, and as a result, the second protective film 114 is easily cracked due to thermal shock and loses a protection function.

Optionally, a material of the second protective film 114 is conductive ceramic or metal. Compared with a case that the first protective film 113 is made of a non-conductive material, the second protective film 114 is made of a conductive material, so that the second protective film 114 does not affect the electrical connection between the connection portion 1122 and the power supply assembly 2 while protecting the connection portion 1122 from corrosion of the aerosol-generation substrate. Because the material of the heating material layer 112 is metal, a thermal expansion coefficient of conductive ceramic or metal matches the metal heating material layer 112, and adhesion of conductive ceramic or metal matches the metal heating material layer 112. Therefore, conductive ceramic or metal is used as the second protective film 114, and the second protective film 114 can hardly fall off the connection portion 1122, so that the connection portion can be well protected. Conductive ceramic or metal is used as the second protective film 114, which helps reduce contact resistance.

When the material of the second protective film 114 is conductive ceramic, a material of the conductive ceramic is one or more of titanium nitride or titanium diboride. It may be understood that, conductive ceramic is more resistant to corrosion of the aerosol-generation substrate than metal.

It should be noted that, the connection portion 1122 of the heating material layer 112 and the second protective film 114 form an electrode; and the second protective film 114 is arranged on the connection portion 1122, which reduces resistance and may serve as an electrode. The thickness of the heating portion 1121 and the thickness of the connection portion 1122 may be the same or may be different. To reduce a resistance value of the connection portion 1122, the thickness of the connection portion 1122 may also be greater than that of the heating portion 1121.

Still referring to FIG. 3a and FIG. 4, in this embodiment, the liquid guiding substrate 111 is a dense liquid guiding substrate; and the liquid guiding substrate 111 includes a plurality of first micropores 1113, and the plurality of first micropores 1113 are ordered through holes running through the first surface 1111 and the second surface 1112. The aerosol-generation substrate in the liquid storage cavity 14 reaches the liquid guiding substrate 111 of the heating body 11 through the liquid supplying channel 1211, and the aerosol-generation substrate is guided from the second surface 1112 of the liquid guiding substrate 111 to the first surface 1111 of the liquid guiding substrate 111 through capillary force of the plurality of first micropores 1113 on the liquid guiding substrate 111, so that the aerosol-generation substrate is vaporized by the heating material layer 112 arranged on the first surface 1111. That is, the plurality of first micropores 1113 are in communication with the liquid storage cavity 14 through the liquid supplying channel 1211. A material of the liquid guiding substrate 111 may be quartz, glass, or dense ceramic, and the plurality of first micropores 1113 are straight through holes in this case; and when the material of the liquid guiding substrate 111 is glass, the glass may be one of common glass, quartz glass, borosilicate glass, or photosensitive lithium aluminosilicate glass.

Optionally, the plurality of first micropores 1113 are only provided in the heating region a of the liquid guiding substrate 111, and no first micropore 1113 is provided in the electrode region b. In an embodiment, the heating portion 1121 is not only arranged on the first surface 1111, and is further arranged on an inner surface of each of the plurality of first micropores 1113. The second protective film 114 is also arranged in each of the plurality of first micropores 1113 and totally covers the heating portion 1121 arranged on the inner surface of each of the plurality of first micropores 1113.

It may be understood that, when the power of the electronic vaporization device ranges from 6 W to 8.5 W and the voltage of the battery ranges from 2.5 V to 4.4 V, to reach operating resistance of the battery, the resistance of the heating material layer 112 of the heating body 11 at normal temperature ranges from 0.5Ω to 2Ω In this embodiment, the heating material layer 112 covers the entire heating region a.

In this application, the plurality of first micropores 1113 including capillary force are provided on the liquid guiding substrate 111, so that a porosity of the heating body 11 can be accurately controlled, thereby improving the product consistency. That is, in batch production, the porosity of the liquid guiding substrate 111 in the heating body 11 is basically consistent, and the thickness of the heating material layer 112 formed on the liquid guiding substrate 111 is uniform, so that vaporization effects of electronic vaporization devices produced in one batch are consistent.

Compared with an existing cotton core heating body and a porous ceramic heating body, the heating body 11 in a thin-sheet structure and provided with the plurality of first micropores 1113 provided in this application has a shorter liquid supplying channel and a fast liquid supplying speed, but also has a larger risk of liquid leakage. Therefore, the inventor of this application researched an impact of a ratio of the thickness of the liquid guiding substrate 111 to a pore size of each of the plurality of first micropores 1113 on liquid supplying of the heating body 11, and found that the risk of liquid leakage may be reduced by increasing the thickness of the liquid guiding substrate 111 and reducing the pore size of each of the plurality of first micropore 1113 but a liquid supplying rate may also be reduced, and the liquid supplying rate may be increased by reducing the thickness of the liquid guiding substrate 111 and increasing the pore size of each of the plurality of first micropores 1113 but the risk of liquid leakage may also be increased, which conflict with each other. Therefore, in this application, the thickness of the liquid guiding substrate 111, the pore size of each of the plurality of first micropores 1113, and the ratio of the thickness of the liquid guiding substrate 111 to the pore size of each of the plurality of first micropores 1113 are designed, so that sufficient liquid supplying can be implemented while liquid leakage is prevented when the heating body 11 works under conditions that the power ranges from 6 W to 8.5 W and the voltage ranges from 2.5 V to 4.4 V. The thickness of the liquid guiding substrate 111 is a distance between the first surface 1111 and the second surface 1112.

In addition, the inventor of this application further researched a ratio of a distance between centers of adjacent first micropores 1113 to the pore size of each of the plurality of first micropores 1113, and found that if the ratio of the distance between centers of adjacent first micropores 1113 to the pore size of each of the plurality of first micropores 1113 is excessively great, the liquid guiding substrate 111 has relatively great intensity and is easy to manufacture, but a porosity is excessively small, which easily leads to an insufficient liquid supplying amount; and if the ratio of the distance between centers of adjacent first micropores 1113 to the pore size of each of the plurality of first micropores 1113 is excessively small, the porosity is relatively great, so that the liquid supplying amount is sufficient, but the liquid guiding substrate 111 has relatively small intensity and is hard to manufacture. Therefore, in this application, the ratio of the distance between centers of adjacent first micropores 1113 to the pore size of each of the plurality of first micropores 1113 is further designed, so that the intensity of the liquid guiding substrate 111 is improved as much as possible while the liquid supplying capability is met.

A description is provided below by using an example in which the material of the liquid guiding substrate 111 is glass.

Specifically, both the first surface 1111 and the second surface 1112 include a smooth surface, and the first surface 1111 is a flat surface. That is, the first surface 1111 of the liquid guiding substrate 111 is a smooth flat surface. The first surface 1111 being a smooth flat surface is conducive to deposition and film formation of a metal material with a relatively small thickness, that is, conducive to formation of the heating material layer 112 on the first surface 1111 of the liquid guiding substrate 111.

Optionally, both the first surface 1111 and the second surface 1112 of the liquid guiding substrate 111 are smooth flat surfaces, and the first surface 1111 and the second surface 1112 of the liquid guiding substrate 111 are arranged opposite to each other; and an axis of each of the plurality of first micropores 1113 is perpendicular to the first surface 1111 and the second surface 1112, and the thickness of the liquid guiding substrate 111 is equal to a length of each of the plurality of first micropores 1113. It may be understood that, the second surface 1112 is parallel to the first surface 1111, and the plurality of first micropores 1113 run through from the first surface 1111 to the second surface 1112, so that a production process of the liquid guiding substrate 111 is simple and costs are reduced. The distance between the first surface 1111 and the second surface 1112 is the thickness of the liquid guiding substrate 111.

Optionally, the first surface 1111 of the liquid guiding substrate 111 is a smooth flat surface; and the second surface 1112 of the liquid guiding substrate 111 is a smooth non-flat surface such as an inclined surface, a cambered surface, or a serrated surface, and the second surface 1112 may be designed according to a specific requirement, provided that the plurality of first micropores 1113 run through the first surface 1111 and the second surface 1112.

Optionally, a cross section of each of the plurality of first micropores 1113 is in shape of a circle. The plurality of first micropores 1113 may be straight through holes with a uniform pore size or may be straight through holes with a non-uniform pore size, provided that a change range of the pore size falls within 50%. For example, due to limitation of a preparation process, in a first micropore 1113 provided on the glass through laser induction and corrosion, a pore size at two ends is generally greater than a pore size at a middle part. Therefore, it is only required to ensure that the pore size at the middle part of the first micropore 1113 to be not less than a half of the pore size at the two ends.

The following describes thickness of the liquid guiding substrate 111, the pore size of each of the plurality of first micropores 1113, the ratio of the thickness of the liquid guiding substrate 111 to the pore size of each of the plurality of first micropores 1113, and the ratio of the distance between centers of adjacent first micropores 1113 to the pore size of each of the plurality of first micropores 1113 by using an example in which the material of the liquid guiding substrate 111 is glass and both the first surface 1111 and the second surface 1112 of the liquid guiding substrate 111 are smooth flat surfaces and are arranged parallel to each other.

The thickness of the liquid guiding substrate 111 ranges from 0.1 mm to 1 mm. When the thickness of the liquid guiding substrate 111 is greater than 1 mm, the liquid supplying requirement cannot be met, leading to a decrease in the amount of aerosols, a great heat loss, and high costs for providing the plurality of first micropores 1113; and when the thickness of the liquid guiding substrate 111 is less than 0.1 mm, the intensity of the liquid guiding substrate 111 cannot be ensured, which is not conducive to improve the performance of the electronic vaporization device. Optionally, the thickness of the liquid guiding substrate 111 ranges from 0.2 mm to 0.5 mm. It may be understood that, the thickness of the liquid guiding substrate 111 is selected according to an actual requirement.

The pore size of each of the plurality of first micropores 1113 on the liquid guiding substrate 111 ranges from 1 μm to 100 μm. When the pore size of each of the plurality of first micropores 1113 is less than 1 μm, the liquid supplying requirement cannot be met, leading to a decrease in an amount of aerosols; and when the pore size of each of the plurality of first micropores 1113 is greater than 100 μm, the aerosol-generation substrate may easily leak out from the plurality of first micropores 1113 to the first surface 1111 to cause liquid leakage, leading to a decrease in the vaporization efficiency. Optionally, the pore size of each of the plurality of first micropores 1113 ranges from 20 μm to 50 μm. It may be understood that, the pore size of each of the plurality of first micropores 1113 is selected according to an actual requirement.

Optionally, the ratio of the thickness of the liquid guiding substrate 111 to the pore size of each of the plurality of first micropores 1113 ranges from 20:1 to 3:1. Optionally, the ratio of the thickness of the liquid guiding substrate 111 to the pore size of each of the plurality of first micropores 1113 ranges from 15:1 to 5:1. When the ratio of the thickness of the liquid guiding substrate 111 to the pore size of each of the plurality of first micropores 1113 is greater than 20:1, the aerosol-generation substrate supplied through the capillary force of each of the plurality of first micropores 1113 can hardly meet a vaporization required amount of the heating body 11, which easily leads to dry burning and a decrease in an amount of aerosols generated in single vaporization; and when the ratio of the thickness of the liquid guiding substrate 111 to the pore size of each of the plurality of first micropores 1113 is less than 3:1, the aerosol-generation substrate may easily leak out from each of the plurality of first micropores 1113 to the first surface 1111 to cause a waste of the aerosol-generation substrate, leading to a decrease in the vaporization efficiency and a decrease in a total amount of aerosols.

The ratio of the distance between centers of two adjacent first micropores 1113 to the pore size of each of the plurality of first micropores 1113 ranges from 3:1 to 1.5:1, so that the intensity of the liquid guiding substrate 111 is improved as much as possible while causing the plurality of first micropores 1113 on the liquid guiding substrate 111 to meet the liquid supplying capability. Optionally, the ratio of the distance between centers of two adjacent first micropores 1113 to the pore size of each of the plurality of first micropores 1113 ranges from 3:1 to 2:1. Optionally, the ratio of the distance between centers of two adjacent first micropores 1113 to the pore size of each of the plurality of first micropores 1113 ranges from 3:1 to 2.5:1.

In a specific embodiment, the ratio of the thickness of the liquid guiding substrate 111 to the pore size of each of the plurality of first micropores 1113 ranges from 15:1 to 5:1, and the ratio of the distance between centers of two adjacent first micropores 1113 to the pore size of each of the plurality of first micropores 1113 ranges from 3:1 to 2.5:1.

In this embodiment, the liquid guiding substrate 111 is in a shape of a flat plate. For example, the liquid guiding substrate 111 is in a shape of a rectangular plate or a circular plate, which is specifically designed as required. In some other implementations, the liquid guiding substrate 111 is in a shape of an arc or a barrel. The plurality of first micropores 1113 are arranged in an array in the heating region a. That is, the plurality of first micropores 1113 provided on the liquid guiding substrate 111 are regularly arranged, and distances between centers of adjacent first micropores 1113 among the plurality of first micropores 1113 are the same. The pore sizes of the plurality of first micropores 1113 may be the same or may be different, which is designed as required.

The liquid guiding substrate 111 in the heating body 11 is a dense material, so that the liquid guiding substrate can play a role of structure supporting. Compared with a spring-shaped metal heating wire in the existing cotton core heating body or a metal thick-film wire in the porous ceramic heating body, the intensity and the thickness of the heating material layer 112 in the heating body 11 are not required, and the heating material layer 112 may adopt a metal material with a low resistivity such as gold or aluminum.

In an implementation, the heating material layer 112 formed on the first surface 1111 of the liquid guiding substrate 111 is a heating film, and the thickness of the heating material layer 112 ranges from 200 nm to 5 μm, namely, the thickness of the heating material layer 112 is relatively small. Optionally, the thickness of the heating material layer 112 ranges from 200 nm to 1 μm. Optionally, the thickness of the heating material layer 112 ranges from 200 nm to 500 nm. When the heating material layer 112 is a heating film, a plurality of second micropores 1123 corresponding to the plurality of first micropores 1113 are provided on the heating material layer 112. Further, the heating material layer 112 is further formed on an inner surface of each of the plurality of first micropores 1113. Optionally, the heating material layer 112 is further formed on the entire inner surface of each of the plurality of first micropores 1113. The heating material layer 112 is arranged on the inner surface of each of the plurality of first micropores 1113, so that the aerosol-generation substrate can be vaporized in the plurality of first micropores 1113, thereby helping improve a vaporization effect.

It may be understood that, when the thickness of the heating material layer 112 is greater than 5 μm, the heating material layer 112 is generally formed in a printing manner, and the plurality of first micropores 1113 may be blocked if the thickness of the heating material layer 112 is excessively great; and the thickness of the heating material layer 112 may range from 5 μm to 100 μm. In this embodiment, the heating material layer 112 covers the entire heating region a, and to prevent the liquid supplying from being affected, the thickness of the heating material layer 112 is not greater than 5 μm.

Optionally, a resistivity of the heating material layer 112 is not greater than 0.06*10⁻⁶-Ω·m. On the basis that the resistance of the heating material layer 112 at normal temperature ranges from 0.5Ω to 2Ω, in this application, a metal material with a low electrical conductivity is used to form a relatively thin metal film, so that the impact on the pore size of each of the plurality of first micropores 1113 is reduced as much as possible, A thinner heating material layer 112 indicates a smaller impact on the pore size of each of the plurality of first micropores 1113 and a better vaporization effect. In addition, a thinner heating material layer 112 indicates a small amount of heat absorbed by the heating material layer 112, lower electric and heat losses, and a fast heat rising temperature of the heating body 11.

Optionally, the metal material of the heating material layer 112 includes silver and alloy thereof, copper and alloy thereof, aluminum and alloy thereof, gold and alloy thereof, nickel and alloy thereof, chromium and alloy thereof, platinum and alloy thereof, titanium and alloy thereof, zirconium and alloy thereof, palladium and alloy thereof, or iron and alloy thereof. In an implementation, the material of the heating material layer 112 may include aluminum and alloy thereof and gold and alloy thereof. Because the liquid aerosol-generation substrate includes various flavors and fragrances and additives and elements such as sulphur, phosphorus, or chlorine, gold include quite strong chemical inertness and a dense oxide thin film may be generated on a surface of aluminum, so that the two materials are quite stable in the liquid aerosol-generation substrate, and are preferably selected as the material of the heating material layer 112.

Optionally, the heating material layer 112, the first protective film 113, and the second protective film 114 may be formed on the first surface 1111 of the liquid guiding substrate 111 in a physical vapor deposition (for example, magnetron sputtering, vacuum evaporation, or ion plating) or a chemical vapor deposition (plasma-assisted chemical deposition, laser-assisted chemical deposition, or metal organic compound deposition) manner.

It may be understood that, due to formations process of the heating material layer 112 and the first protective film 113, the plurality of first micropores 1113 may not be covered by the heating material layer and the first protective film. The heating material layer 112 and the first protective film 113 extend into a wall surface of each of the plurality of first micropores 1113. While the heating material layer 112 and the first protective film 113 are formed on the first surface 1111 of the liquid guiding substrate 111 in a physical vapor deposition or chemical vapor deposition manner, the heating material layer 112 and the first protective film 113 are also formed on the inner surface of each of the plurality of first micropores 1113. When the heating material layer 112 and the first protective film 113 are formed on the first surface 1111 of the liquid guiding substrate 111 in a magnetron sputtering manner, metal atoms during magnetron sputtering are perpendicular to the first surface 1111 and are parallel to the inner surface of each of the plurality of first micropores 1113, so that the metal atoms are more easily deposited on the first surface 1111. It is assumed that the thickness of the heating material layer 112 and the first protective film 113 formed by the metal atoms deposited on the first surface 1111 is 1 μm, in this case, a thickness of the metal atoms deposited on the inner surface of each of the plurality of first micropores 1113 is far less than 1 μm and even less than 0.5 μm. A smaller thickness of the heating material layer 112 and the first protective film 113 deposited on the first surface 1111 indicates a smaller thickness of the heating material layer 112 and the first protective film 113 formed on the inner surface of each of the plurality of first micropores 1113 and a smaller impact on the pore size of each of the plurality of first micropores 1113. Because the thickness of the heating material layer 112 and the first protective film 113 is far less than the pore size of each of the plurality of first micropores 1113, and the thickness of the part of the heating material layer 112 and the first protective film 113 deposited in each of the plurality of first micropores 1113 is less than the thickness of the part deposited on the first surface 1111 of the liquid guiding substrate 111, the impact of the heating material layer 112 and the first protective film 113 deposited in each of the plurality of first micropores 1113 on the pore size of each of the plurality of first micropores 1113 may be omitted.

In some other implementations, the material of the liquid guiding substrate 111 is porous ceramic, a plurality of capillary holes that are interconnected and disorderly distributed are provided in the porous ceramic, the liquid guiding is performed by using the capillary holes of the porous ceramic. That is, the liquid guiding substrate 111 includes a plurality of disordered through holes. The first protective film 113 is arranged on the heating portion 1121 of the heating material layer 112, and the second protective film 114 is arranged on the connection portion 1122 of the heating material layer 112, so that the heating material layer 112 is protected. That is, the first protective film 113 and the second protective film 114 provided in this application may be applicable to a surface of a conventional porous ceramic heating body, to protect a heating material layer thereof.

Referring to FIG. 5, FIG. 5 is a schematic structural diagram of a second implementation of a heating body according to this application. The liquid guiding substrate 111 may also be composite ceramic. The liquid guiding substrate 111 includes a porous ceramic layer and a dense ceramic layer that are stacked, the dense ceramic layer includes a plurality of ordered straight through holes perpendicular to a thickness direction of the liquid guiding substrate 111; and the heating material layer 112 is arranged on a surface of the dense ceramic layer that is away from the porous ceramic layer. Specifically, the liquid guiding substrate 111 includes a first liquid guiding substrate 111a and a second liquid guiding substrate 111b, namely, the first liquid guiding substrate 111a is a porous ceramic layer, and the second liquid guiding substrate 111b is a dense ceramic layer. A surface of the first liquid guiding substrate 111a that is away from the second liquid guiding substrate 111b is the second surface 1112 of the liquid guiding substrate 111, and a surface of the second liquid guiding substrate 111b that is away from the first liquid guiding substrate 111a is the first surface 1111. A material of the first liquid guiding substrate 111a is porous ceramic, and the first liquid guiding substrate 111a includes a plurality of disordered through holes; a material of the second liquid guiding substrate 111b is dense ceramic, the second liquid guiding substrate 111b includes a plurality of first micropores 1113, the plurality of first micropores 1113 are run-through holes, and an axis of each of the plurality of first micropores 1113 is parallel to a thickness direction of the second liquid guiding substrate 111b; and the heating material layer 112 is arranged on a surface of the second liquid guiding substrate 111b that is away from the first liquid guiding substrate 111a. The first protective film 113 is arranged on the heating portion 1121 of the heating material layer 112, and the second protective film 114 is arranged on the connection portion 1122 of the heating material layer 112, so that the heating material layer 112 is protected.

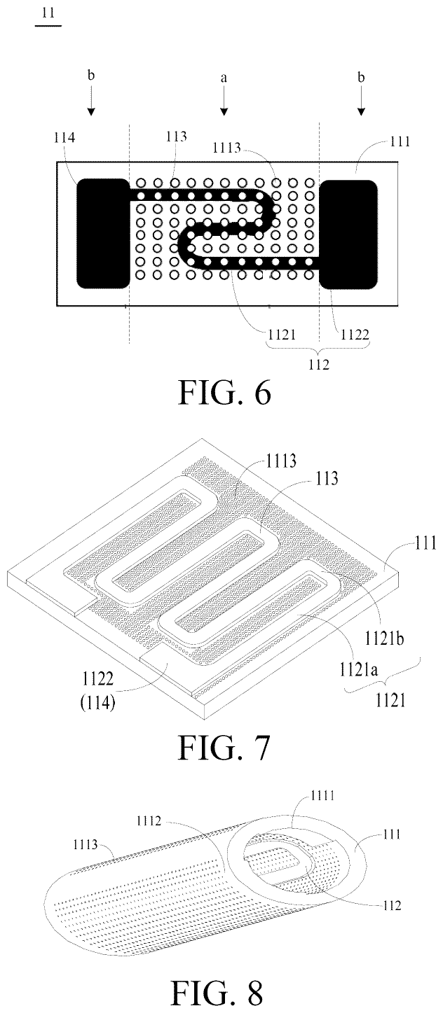

Referring to FIG. 6, FIG. 6 is a schematic structural diagram of a third implementation of a heating body according to this application.

A difference between the heating body 11 shown in FIG. 6 and the heating body 11 shown in FIG. 3a lies in that: in FIG. 3a, the heating material layer 112 covers the entire heating region a or crosses the entire heating region a., and in FIG. 6, the heating material layer 112 covers a part of the heating region a, that is, shapes of the heating material layer 112 are different, and other same structures are not described herein again.

As shown in FIG. 6, the heating portion 1121 of the heating material layer 112 is in a shape of a S-shaped bended strip, to form a temperature field with a temperature gradient on the first surface 1111 of the liquid guiding substrate 111, that is, to form a high-temperature region and a low-temperature region on the first surface 1111 of the liquid guiding substrate 111, so as to vaporize various components in the aerosol-generation substrate to the greatest extent. Two ends of the heating portion 1121 are respectively connected to one connection portions 1122. A size of the connection portion 1122 is greater than a size of the heating portion 1121, to help the connection portion 1122 to better implement an electrical connection with the power supply assembly 2. A resistivity of the heating material layer 112 is not greater than 0.06*10⁻⁶ Ω·m. Optionally, the heating portion 1121 and the connection portion 1122 are integrally formed.

The first protective film 113 is arranged on a surface of the heating portion 1121 that is away from the liquid guiding substrate 111, the second protective film 114 is arranged on a surface of the connection portion 1122 that is away from the liquid guiding substrate 111, and for details of the first protective film 113 and the second protective film 114, reference may be made to the foregoing description.

In an implementation, the heating material layer 112 formed on the first surface 1111 of the liquid guiding substrate 111 is a heating film, and the thickness of the heating material layer 112 ranges from 200 nm to 5 μm, namely, the thickness of the heating material layer 112 is relatively small. Optionally, the thickness of the heating material layer 112 ranges from 200 nm to 1 μm. Optionally, the thickness of the heating material layer 112 ranges from 200 nm to 500 nm. Optionally, the heating material layer 112 is formed in a physical vapor deposition (for example, magnetron sputtering, vacuum evaporation, or ion plating) or a chemical vapor deposition (plasma-assisted chemical deposition, laser-assisted chemical deposition, or metal organic compound deposition) manner.

In another implementation, the thickness of the heating material layer 112 formed on the first surface 1111 of the liquid guiding substrate 111 ranges from 5 μm to 100 μm, namely, the thickness of the heating material layer 112 is relatively great. Optionally, the thickness of the heating material layer 112 ranges from 5 μm to 50 μm. Optionally, the heating material layer 112 is formed on the first surface 1111 of the liquid guiding substrate 111 in a printing manner, namely, the heating material layer 112 is a printed metal slurry layer. Because the first surface 1111 of the liquid guiding substrate 111 has a low degree of roughness, a consecutive film shape may be formed when the thickness of the heating material layer 112 is less than 100 μm.

It may be understood that, the heating material layer 112 in FIG. 6 covers a part of the heating region a, and the thickness of the heating material layer 112 may be set to range from 5 μm to 100 μm, even if a region where the heating material layer 112 is arranged blocks a part of the plurality of first micropores 1113, liquid supplying may still be performed by other first micropores 1113. The liquid guiding substrate 111 of the heating body 11 shown in FIG. 6 may be a dense liquid guiding substrate, porous ceramic, or composite ceramic (the liquid guiding substrate 111 shown in FIG. 5).

Referring to FIG. 7, FIG. 7 is a schematic structural diagram of a fourth implementation of a heating body according to this application.

A difference between the heating body 11 shown in FIG. 7 and the heating body 11 shown in FIG. 3a lies in that: shapes of the heating material layer 112 are different, and other same structures are not described herein again.

As shown in FIG. 7, the liquid guiding substrate 111 is in a shape of a flat plate, the heating portion 1121 of the heating material layer 112 includes a plurality of first heating portions 1121a extending in a first direction and a plurality of second heating portions 1121b extending in a second direction, and each of the plurality of second heating portions 1121b connects two adjacent first heating portions 1121a. Two connection portions 1122 are arranged on the same side of the heating portion 1121. A width of the connection portion 1122 is greater than a width of the heating portion 1121.

The first protective film 113 is arranged on a surface of the heating portion 1121 that is away from the liquid guiding substrate 111, the second protective film 114 is arranged on a surface of the connection portion 1122 that is away from the liquid guiding substrate 111, and for details of the first protective film 113 and the second protective film 114, reference may be made to the foregoing description.

Referring to FIG. 8, FIG. 8 is a schematic structural diagram of a fifth implementation of a heating body according to this application.

A difference between the heating body 11 shown in FIG. 8 and the heating body 11 shown in FIG. 3a lies in that: shapes of the heating body 11 are different, and other same structures are not described herein again.

As shown in FIG. 8, the liquid guiding substrate 111 is in a shape of a barrel, the liquid guiding substrate 111 is a dense liquid guiding substrate, the liquid guiding substrate 111 includes a plurality of first micropores 1113, and the plurality of first micropores 1113 are straight through holes running through the first surface 1111 and the second surface 1112. The first surface 1111 is an inner surface of the barrel-shaped liquid guiding substrate 111, and the second surface 1112 is an outer surface of the barrel-shaped liquid guiding substrate 111. The heating material layer 112 is arranged on the first surface 1111 of the liquid guiding substrate 111. The first protective film 113 and the second protective film 114 are arranged on a surface of the heating material layer 112 that is away from the liquid guiding substrate 111. It should be noted that, the first protective film 113 and the second protective film 114 are not marked in FIG. 8.

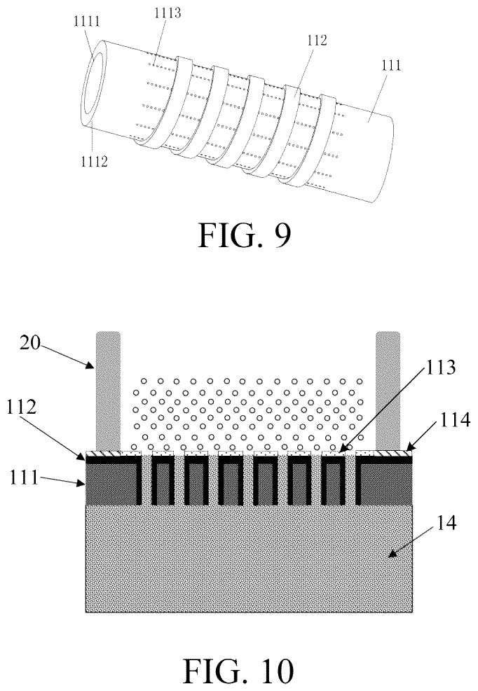

Referring to FIG. 9, FIG. 9 is a schematic structural diagram of a sixth embodiment of a heating body according to this application.

A difference between the heating body 11 shown in FIG. 9 and the heating body 11 shown in FIG. 3a lies in that: shapes of the heating body 11 are different, and other same structures are not described herein again.

As shown in FIG. 9, the liquid guiding substrate 111 is in a shape of a barrel, the liquid guiding substrate 111 is a dense liquid guiding substrate, the liquid guiding substrate 111 includes a plurality of first micropores 1113, and the plurality of first micropores 1113 are straight through holes running through the first surface 1111 and the second surface 1112. The first surface 1111 is an inner surface of the barrel-shaped liquid guiding substrate 111, and the second surface 1112 is an outer surface of the barrel-shaped liquid guiding substrate 111. The heating material layer 112 is arranged on the second surface 1112 of the liquid guiding substrate 111. The first protective film 113 and the second protective film 114 are arranged on a surface of the heating material layer 112 that is away from the liquid guiding substrate 111. It should be noted that, the first protective film 113 and the second protective film 114 are not marked in FIG. 9.

The following verifies a relationship among the material of the heating material layer 112, the material of the first protective film 113, the material of the second protective film 114 and a service life of the heating body 11 and a relationship among the material of the first protective film 113, the material of the second protective film 114, and a vaporization amount through experiments. Referring to FIG. 10, FIG. 10 is a schematic diagram of wet combustion on a heating body according to this application.

Experiment one: A cartridge is loaded in the heating body 11 and wet combustion is performed to evaluate the service life of the heating body 11. Experiment conditions: a mode that energy is supplied at constant power of 6.5 W and inhalation is performed for 3 seconds and stopped for 27 seconds is used, and the aerosol-generation substrate is 30 mg cola ice. The heating body 11 is set to compare a case provided with the first protective film 113 and a case not provided with a protective film, the first protective film 113 selects different materials for comparison, and experiments are performed by simulating a normal use environment of the electronic vaporization device (referring to FIG. 10). A comparison result is shown in Table 3, and a relationship among the material of the heating material layer 112, the material of the first protective film 113, and the service life of the heating body 11 is obtained. In FIG. 10, energy is supplied by using a direct current power supply, and ejector pins 20 of the power supply assembly 2 (the ejector pins 20 are electrically connected to a battery) are respectively connected to the connection portions 1122 of the heating material layer 112, to control powered-on power and a powered-on time.

TABLE 3
Relationship among the material of the heating material layer, the material
of the first protective film, and a service life of the heating body

| Heating | Protective film | | | | | |
|---|---|---|---|---|---|---|
| material | | 316L stainless | Aluminum | Silicon | Aluminum | Silicon |
| layer | None | steel | nitride | nitride | oxide | carbide |
| Silver | about 30 | >200 | about 300 | about 320 | about 350 | about 250 |
| Copper | about 80 | >200 | about 400 | about 400 | about 400 | about 400 |
| Aluminum | >600 | >750 | >1500 | >1500 | >1500 | >1500 |

When the first protective film 113 is not arranged, materials such as silver and copper serving as the heating material layer 112 are easily corroded by the flavors and fragrances and additives including elements such as sulphur, phosphorus, or chlorine in the aerosol-generation substrate, which can hardly meet a requirement of the service life. When aluminum serves as the material of the heating material layer 112, over 600 times of thermal cycling can be bore, so that a use condition of a closed electronic vaporization device is met, but a requirement of over 1500 puffs of an open electronic vaporization device can be hardly met.

Therefore, the first protective film 113 is arranged on a surface of the heating material layer 112 to improve the service life thereof. The material of the first protective film 113 is a ceramic material resistant to corrosion of the aerosol-generation substrate, such as aluminum nitride, silicon nitride, aluminum oxide, silicon oxide, silicon carbide, or zirconium oxide. No matter the material of the heating material layer 112 is silver, copper, or aluminum, the service life of the heating body 11 can all be greatly improved after the first protective film 113 is adopted.

Experiment two: A cartridge is loaded in the heating body 11 and wet combustion is performed to evaluate the service life of the heating body 11. Experiment conditions: a mode that energy is supplied at constant power of 6.5 W and inhalation is performed for 3 seconds and stopped for 27 seconds is used, and the aerosol-generation substrate is 30 mg cola ice. The heating body 11 is set to compare vaporization amounts of first protective films 113 made of different materials, and experiments are performed by simulating a normal use environment of the electronic vaporization device (referring to FIG. 10). A comparison result is shown in Table 4, and a relationship between the material of the first protective film 113 and the vaporization amount is obtained.

In FIG. 10, energy is supplied by using a direct current power supply, and ejector pins 20 of the power supply assembly 2 (the ejector pins 20 are electrically connected to a battery) are respectively connected to the connection portions 1122 of the heating material layer 112, to control powered-on power and a powered-on time.

TABLE 4
Relationship between the material of the first
protective film and the vaporization amount

| | Protective layer | | |
|---|---|---|---|
| Heating film | 316L stainless steel | Aluminum nitride | Silicon nitride |
| Aluminum | 6.2 mg/puff | 7.2 mg/puff | 6.9 mg/puff |

As can be known from FIG. 4, the vaporization amount is apparently improved when the material of the first protective film 113 selects a ceramic material (for example, aluminum nitride or silicon nitride) when compared with a metal material (for example, 316L stainless steel).

The foregoing descriptions are merely implementations of this application, and the patent scope of this application is not limited thereto. All equivalent structure or process changes made according to the content of this specification and the accompanying drawings in this application or by directly or indirectly applying this application in other related technical fields shall fall within the protection scope of this application.

What is claimed is:

1. A heating body for an electronic vaporization device and configured to vaporize an aerosol-generation substrate, the heating body comprising:
a liquid guiding substrate comprising a heating region and an electrode region;
a heating material layer arranged on a first surface of the liquid guiding substrate, wherein the heating material layer is a resistance heating material and comprises a heating portion arranged in the heating region and a connection portion arranged in the electrode region;
a first protective film at least partially arranged on a surface of the heating portion that is away from the liquid guiding substrate, wherein the first protective film is made of a non-conductive material resistant to a corrosion of the aerosol-generation substrate; and
a second protective film at least partially arranged on a surface of the connection portion that is away from the liquid guiding substrate, wherein the second protective film is made of a conductive material resistant to a corrosion of the aerosol-generation substrate.

2. The heating body according to claim 1, wherein the first protective film is made of at least one of ceramic or glass.

3. The heating body according to claim 2, wherein the first protective film is made of one or more of aluminum nitride, silicon nitride, aluminum oxide, silicon oxide, silicon carbide, or zirconium oxide.

4. The heating body according to claim 1, wherein a thickness of the first protective film ranges from 10 nm to 1000 nm.

5. The heating body according to claim 1, wherein the second protective film is made of conductive ceramic or metal.

6. The heating body according to claim 5, wherein the second protective film is made of one or more of titanium nitride or titanium diboride.

7. The heating body according to claim 1, wherein a thickness of the second protective film ranges from 10 nm to 2000 nm.

8. The heating body according to claim 1, wherein the liquid guiding substrate is a dense liquid guiding substrate comprising a second surface arranged opposite to the first surface, and a plurality of first micropores that are ordered through holes running through the first surface and the second surface.

9. The heating body according to claim 8, wherein the liquid guiding substrate is made of at least one of quartz, glass, or dense ceramic, and the plurality of first micropores are straight through holes.

10. The heating body according to claim 1, wherein the liquid guiding substrate is made of porous ceramic and comprises a plurality of disordered through holes; or
the liquid guiding substrate comprises a porous ceramic layer and a dense ceramic layer that are stacked, the dense ceramic layer comprises a plurality of ordered straight through holes perpendicular to a thickness direction of the liquid guiding substrate, and the heating material layer is arranged on a surface of the dense ceramic layer that is away from the porous ceramic layer.

11. The heating body according to claim 1, wherein the heating material layer is a heating film, and a thickness of the heating film ranges from 200 nm to 5 μm.

12. The heating body according to claim 11, wherein a resistivity of the heating material layer is less than 0.06*10⁻⁶ Ω·m.

13. The heating body according to claim 11, wherein the heating material layer is made of at least one of aluminum, copper, silver, gold, nickel, chromium, platinum, titanium, zirconium, palladium, iron, or alloy thereof.

14. The heating body according to claim 1, wherein a thickness of the heating material layer ranges from 5 μm to 100 μm, and the heating material layer is a printed metal slurry layer.

15. The heating body according to claim 1, wherein the liquid guiding substrate has a shape of a flat plate, an arc, or a barrel.

16. The heating body according to claim 1, wherein the first protective film covers the heating portion, and the second protective film covers the connection portion.

17. The heating body according to claim 1, wherein the liquid guiding substrate has a shape of a cylinder and comprises an inner surface and an outer surface, and the heating material layer is arranged on the inner surface or the outer surface.

18. The heating body according to claim 1, wherein the heating material layer, the first protective film, and the second protective film are formed on the first surface of the liquid guiding substrate in a physical vapor deposition or chemical vapor deposition manner.

19. The heating body according to claim 1, wherein the connection portion of the heating material layer and the second protective film form an electrode.

20. The heating body according to claim 8, wherein the plurality of first micropores are straight through holes, and the heating material layer and the first protective film extend into a wall surface of each of the plurality of first micropores.

21. A vaporization assembly, comprising:
a liquid storage cavity configured to store a liquid aerosol-generation substrate; and
a heating body in fluid communication with the liquid storage cavity, the heating body comprising:
a liquid guiding substrate comprising a heating region and an electrode region;
a heating material layer arranged on a first surface of the liquid guiding substrate, wherein the heating material layer is a resistance heating material and comprises a heating portion arranged in the heating region and a connection portion arranged in the electrode region;
a first protective film at least partially arranged on a surface of the heating portion that is away from the liquid guiding substrate, wherein the first protective film is made of a non-conductive material resistant to a corrosion of the aerosol-generation substrate; and
a second protective film at least partially arranged on a surface of the connection portion that is away from the liquid guiding substrate, wherein the second protective film is made of a conductive material resistant to a corrosion of the aerosol-generation substrate.

22. An electronic vaporization device, comprising:
a vaporization assembly; and
a power supply assembly,
wherein the vaporization assembly comprises:
a liquid storage cavity configured to store a liquid aerosol-generation substrate; and
a heating body in fluid communication with the liquid storage cavity, the heating body comprising:
a liquid guiding substrate comprising a heating region and an electrode region;
a heating material layer arranged on a first surface of the liquid guiding substrate, wherein the heating material layer is a resistance heating material and comprises a heating portion arranged in the heating region and a connection portion arranged in the electrode region;
a first protective film at least partially arranged on a surface of the heating portion that is away from the liquid guiding substrate, wherein the first protective film is made of a non-conductive material resistant to a corrosion of the aerosol-generation substrate; and
a second protective film at least partially arranged on a surface of the connection portion that is away from the liquid guiding substrate, wherein the second protective film is made of a conductive material resistant to a corrosion of the aerosol-generation substrate; and
the power supply assembly is connected to the heating body.
